(19) United States
(12) Patent Application Publication
(10) Pub. No.: US 2013/0211442 A1
(43) Pub. Date: Aug. 15, 2013

(54) METHOD AND APPARATUS FOR CUTTING EMBOLIC COILS

(71) Applicant: Aftab S. Karim, Burton, MI (US)

(72) Inventor: Aftab S. Karim, Burton, MI (US)

(73) Assignee: Neurosurj Research & Development, LLC, Flint, MI (US)

(21) Appl. No.: 13/520,868

(22) PCT Filed: Jan. 7, 2011
(86) PCT No.: PCT/US11/20552

§ 371 Date: Dec. 4, 2012

Related U.S. Application Data
(60) Provisional application No. 61/293,503, filed on Jan. 8, 2010.

(51) Int. Cl. A61B 17/12

(52) U.S. Cl. CPC A61B 17/1214
USPC 606/200

(57) ABSTRACT

The invention relates to a hand held microsurgical apparatus for cutting metallic embolic coils and the methods pertaining thereto.

### CROSS-REFERENCE TO RELATED APPLICATION

The present application claims the benefit under 35 U.S.C. §119(e) of U.S. Provisional Application No. 61/293,503, filed Jan. 8, 2010, which is hereby incorporated by reference in its entirety.

### FIELD OF THE INVENTION

The invention relates to a hand held microsurgical apparatus for cutting metallic embolic coils and the methods pertaining thereto.

### BACKGROUND OF THE INVENTION

Embolic coils are routinely used to treat endovascular malformations in patients around the world in a number of different applications, such as neurological applications and/or peripheral applications. Specifically, they can be used to occlude a vessel, and/or to treat an aneurysm (e.g., an intracranial aneurysm), an arteriovenous malformation (AVM), or a fistula. These types of malformations, when left untreated, can lead to vascular rupture which can then result in neurological damage, complications, or even death. Typically, the treatment procedure involves delivering the embolic coil to the desired vascular treatment site via a microcatheter and deploying a sufficient amount of the embolic coil to fill the weakened portion of the vessel. Once the embolic coil is in place, the body responds by forming a blood clot around the coil, thus reducing the risk of vascular rupture.

When performing a vascular embolization using embolic coils, it may be that the coil, once deployed, does not fit properly within the treatment site. It may also be that open surgical treatment may be needed where embolic coils may need to be trimmed or cut. In addition, if an aneurysm or vascular malformation has been previously treated sub-optimally with metallic embolic coils, the aneurysm or vascular malformation may need to be surgically clipped within the brain or spine of a patient. Such surgical clipping of the aneurysm or vascular malformation may require a device capable of cutting the embolic coils prior to the surgical clipping of the aneurysm or vascular malformation. In some cases, excision of the aneurismal sac may require a cutting device capable of cutting the embolic coils. Ideally in these microsurgical procedures, the cutting would take place both quickly and cleanly in order to minimize the duration of the surgery as well as avoid any potentially dangerous debris from contaminating, and thus potentially damaging, the brain or spine of the patient.

### BRIEF SUMMARY OF THE INVENTION

The apparatus according to the present invention provides a small, hand held device for cleanly cutting embolic coils prior to or during a microsurgical procedure with one hand while avoiding exposure of the patient to unwanted debris. The apparatus disclosed herein provides the user with the maximum leverage using a minimum effort.

The present invention provides methods and an apparatus for cutting coils within the brain or spine of a patient during a microsurgical procedure while minimizing the possibility of damage to the brain or the spine (i.e. blood vessels, nerves, tissue, etc.). The size, shape and design of the cutting blades of the apparatus disclosed herein provides the user with both the strength and dexterity required to perform such a delicate task while in close proximity to delicate brain matter or the spine of a patient undergoing a microsurgical procedure.

Accordingly, one embodiment of the present invention is directed to an apparatus for cutting embolic coils comprising:

first and second legs each having a cutting portion and a connecting portion wherein the legs are pivotally connected at a first pivot point between the cutting portion and the connecting portion;

first and second handles which are pivotally connected at a second pivot point and each is pivotally connected to at least one leg in the connecting portion of the leg at a third and fourth pivot point and wherein all pivot points are substantially planar,

wherein the cutting portion comprises cutting blades on the top and front of the cutting portion such that the cutting portions form a spoon element on the bottom,

wherein the legs are adjustable by the handles between an open position and a closed position and further wherein, in said closed position, the cutting portions are capable of cutting embolic coils.

In one embodiment, the first handle is pivotally connected to the first leg and the second handle is pivotally connected to the second leg at the third and fourth pivot points. In one embodiment, the third and fourth pivot points are between the first and the second pivot points.

Another aspect of the present invention discloses a method for cutting an embolic coil during a microsurgical procedure in the brain or spine of a patient, wherein said method comprises cutting said coil while minimizing the possibility of damage to the brain or spine. The microsurgical procedure is typically performed to treat an aneurysm or a vascular malformation (i.e. arteriovenous malformation, etc.). The microsurgical procedure is typically an open microsurgical procedure such that surgical site within the brain or spine is made available for the device to operate.

Yet another aspect of the present invention discloses a method for cutting an embolic coil using the apparatus disclosed herein by positioning a coil to be cut between the cutting portions of the legs and adjusting the handles such that the legs are in a closed position.

In certain embodiments, the embolic coils are metallic embolic coils. In certain embodiments, the metallic embolic coils comprise platinum, stainless steel, nitinol, and alloys thereof.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 shows one embodiment of an apparatus of the invention.

FIG. 2 shows one embodiment of an top of the apparatus while in the open position.

FIG. 3 shows the bottom of one embodiment of an apparatus while in the open position.

FIG. 4 and FIG. 5 show the bottom of one embodiment of an apparatus while in the open and closed positions, respectively.

FIG. 6 shows a side perspective of one embodiment of an apparatus in the open position.

### DETAILED DESCRIPTION OF THE INVENTION

Before the present apparatus and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

It must be noted that as used herein and in the appended claims, the singular forms “a”, “an”, and “the” include plural referents unless the context clearly dictates otherwise. Thus, for example, reference to “an embolic coil” includes a plurality of various embolic coils and equivalents thereof known to those skilled in the art.

### 1. Definitions

Unless defined otherwise, all technical and scientific terms used herein have the same meaning as commonly understood by one of ordinary skill in the art to which this invention belongs. As used herein the following terms have the following meanings.

As used herein, the term “comprising” or “comprises” is intended to mean that the apparatus and methods include the recited elements, but not excluding others. “Consisting essentially of” when used to define the apparatus and methods, shall mean excluding other elements of any essential significance to the combination for the stated purpose. Thus, an apparatus consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

The term “about” when used before a numerical designation, e.g., temperature, time, amount, and concentration, including range, indicates approximations which may vary by (+) or (−) 10%, 5% or 1%.

“Distal” and “proximal” are intended to refer to the position relative to the user while the apparatus is in use.

As used herein, the terms “embolic coil” and “coil” are intended to refer to a thin wire or wires suitable for use in treating aneurysms or vascular malformations, including but not limited to, coils suitable for treating an aneurysm or a vascular malformations, such as an arteriovenous malformation, a fistula, and the like.

As used herein, the term “vascular malformation” is intended to refer to a blood vessel abnormality such as a arteriovenous malformation (AVM) (e.g. cerebral arteriovenous malformation), a fistula, etc.

As used herein, the phrase “minimizing the possibility of damage” is intended to refer to decreasing the instances of trauma during a microsurgical procedure in the brain or spine (i.e. blood vessels, nerves, tissue, etc.) of the patient. Such trauma that can be avoided with the device and methods disclosed herein might be blunt trauma, which is minimized as the result of an increased dexterity for the user of the device, or penetrating trauma, which is minimized by avoiding debris contamination of the brain or spine as is enabled by the design of the cutting portion of the present invention.

2. Apparatus of the Invention

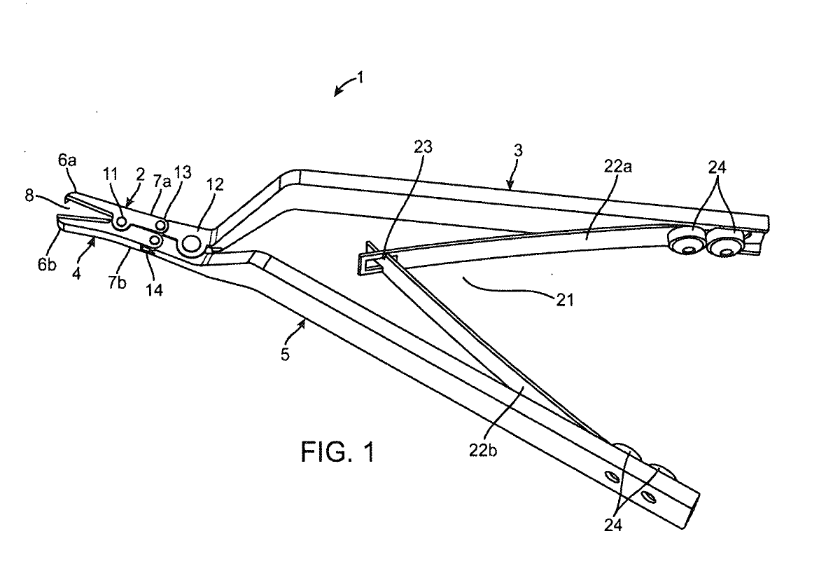

As shown in FIGS. 1, 2 and 3, the apparatus is made up of two leg 2 and 4 portions and two handle 3 and 5 portions. The distal portion of the apparatus 1 is comprised of a first leg 2 and a second leg 4, where each of the legs has a cutting portion (6a and 6b) and a connecting portion (7a and 7b) and the legs 2 and 4 are pivotally connected at a first pivot point 11 between the cutting and connecting portions. The handles of the apparatus 1 comprises first 3 and second 5 handles which are pivotally connected at a second pivot point 12. As can be seen in the Figures, each handle 3 and 5 is pivotally connected to a leg 2 and 4 in the connecting portion of the leg 7a and 7b at a third 13 and fourth 14 pivot points.

FIG. 1 shows the handles 3 and 5 bent upwardly in such a way that the distal portion of the handles are in a plane above, yet substantially parallel to, the cutting and connecting portions of the legs. The bent handle profile allows the user to effectively grip the handles with one hand and precisely position the cutting portions of the legs while preventing the fingers of the user from contaminating and/or touching the surgical site or the embolic coils.

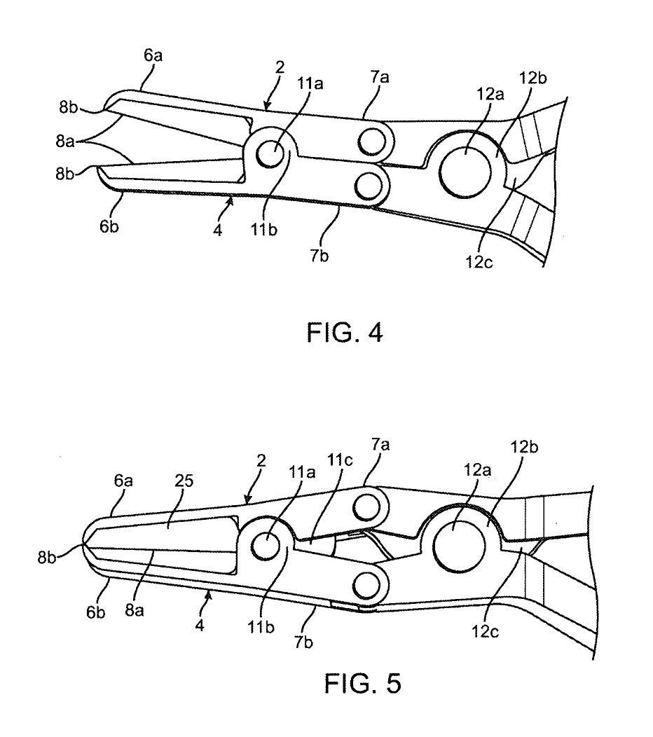

FIGS. 4 and 5 show a bottom view of the distal portion of the apparatus in both the open (FIG. 4) and closed (FIG. 5) positions.

As shown in FIGS. 4 and 5, the first 2 and second 4 legs are pivotally connected at the first pivot point by way of a holding pin 11a. The holding pin 11a secures a lug 11c which is attached to leg 2 between two ears 11b (only one is shown) protruding from leg 4. Similarly, the second pivot point comprises ears 12b and a lug 12c which are pivotally connected such that the top and bottom ears 12b are connected to one handle and the center lug 12c is connected to the other handle and is fitted in between the top and bottom ears 12b. The holding pin 12a is extended through aligned openings in the ears 12b and lug 12c and allows pivotal movement in the horizontal plane. In the apparatus, it is not significant which of the ears or the lugs is connected to which handle or leg (i.e. if ears 11b are on leg 4, then lug 11b must be on leg 2, and vice versa). The other pivot points are designed in a similar fashion.

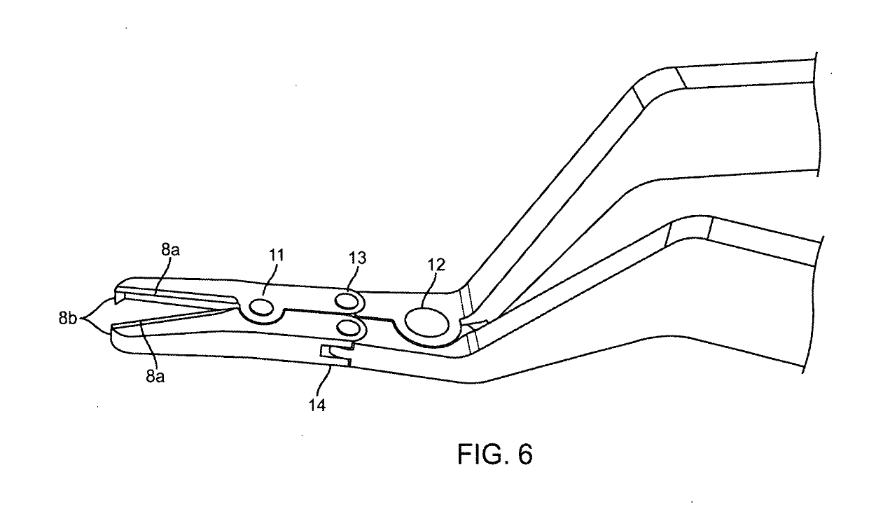

As is shown in FIG. 6, all four of the pivot points are substantially planar with respect to each other. Upon cutting, the first 11 and second 12 pivot points remain substantially longitudinally planar and the third 13 and fourth 14 pair of pivots expand apart from each other and the longitudinal plane of the first 11 and second 12 pivot points.

The embolic coil cutting blades 8 are positioned on the inner edge and distal tip of the cutting portion 6a and 6b of the legs 2 and 4. FIG. 1 shows the apparatus 1 in the open position with the cutting portions 6a and 6b of the legs 2 and 4 apart from each other. The cutting portions 6a and 6b can be closed by adjusting the handles 3 and 5 between an open position and a closed position.

FIG. 4 shows the apparatus in the resting or open position with the cutting portions 6a and 6b of the legs 2 and 4 apart from each other. In this position, the connecting portions 7a and 7b of legs 2 and 4 are touching or are in very close proximity. However, the cutting portion of the first and second legs have a distal portion that is curved such that when the cutting portions 6a and 6b are in contact with each other, the connecting portions (7a and 7b) move apart (FIG. 5).

The cutting portions 6a and 6b are equipped with cutting blades on the top 8a and front 8b of the cutting portion such that the cutting portions form a spoon element 25 on the bottom (FIGS. 4, 5 and 6). The cutting blades are such that a coil can be cut using either the top blades 8a or the front blades 8b allowing the user to have greater freedom in the manipulation of the apparatus. For example, if during a microsurgical procedure the embolic coil requires cutting and space is tight, the doctor is able to cut the coil with the tip of the apparatus. In such embodiments, the apparatus comprises a double bayonet. In addition, the apparatus may have finer, or more slender, tip (6a and 6b) then is depicted in the Figures. Such variations are within the scope of the invention. It is contemplated that the apparatus as disclosed herein, can be used during a microsurgical procedure without the need for multiple cutting devices as the multiple cutting blades of the present apparatus allows the user maneuverability and flexibility.

The handles 3 and 5 are held in the open position when at rest by a spring mechanism 21. Various types of spring mechanisms are known and it is contemplated that any can be used in conjunction with the present invention provided that the force required to manipulate the handles 3 and 5 of the apparatus is not so great that the user cannot easily close the cutting portions 6a and 6b with one hand. As shown in FIG. 1, the spring 21 comprises first 22a and second 22b support arms located interiorly with respect to the handles 3 and 5. One end of the spring support arms 22a and 22b are slidably connected at joint 23 while the opposing end is are fastened 24 to the interior of the handle. The apparatus is shown in the figure to have two spring fasteners 24 (in this case a bolt or screw) secured to each handle 3 and 5, although it is contemplated that one can be used, or alternatively, welding or other attachment means can be implemented. Alternative spring mechanisms are with the scope of the invention and may not require the use of a fastener 24.

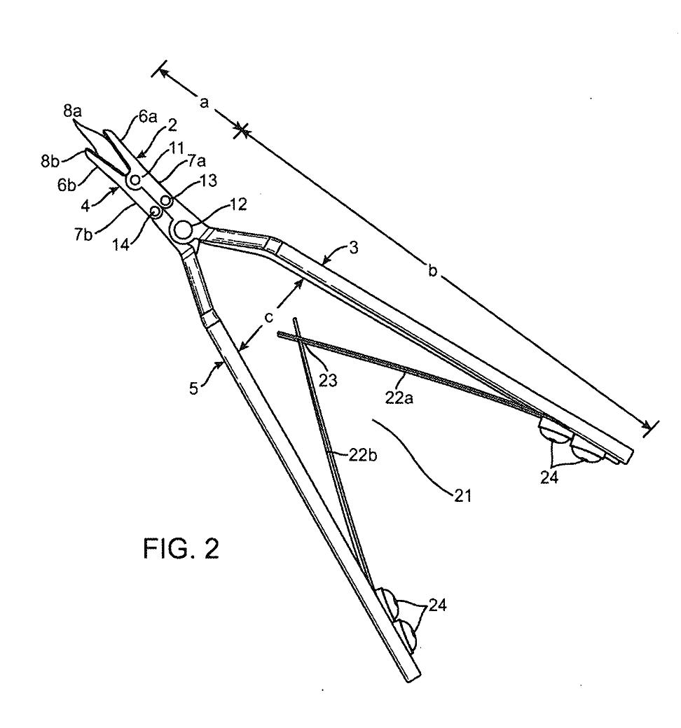

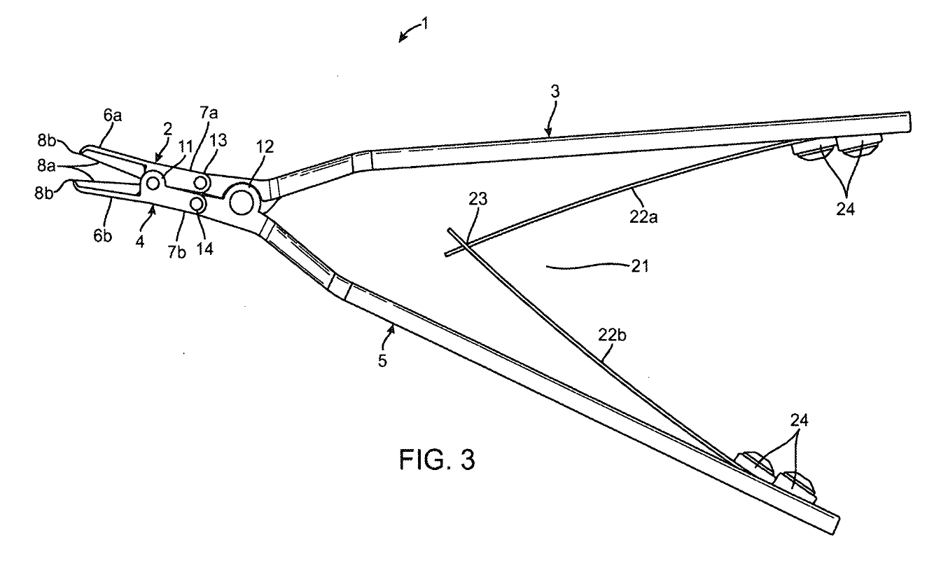

As can be seen in FIGS. 2 and 3, the apparatus 1 is substantially symmetrical so that it can be used by either right or left and users. In addition, the size is such that the apparatus can be used with one hand. For example, the handle portion 3 and 5 of the apparatus (length b in FIG. 2) is from about 3 inches to about 9 inches. In some embodiments, the handle is about 8 inches, or about 7 inches, or about 6 inches, or about 5 inches, or about 4 inches. The cutting legs 2 and 4 (length a in FIG. 2) are from about 1 inch to about 3 inches, or about 2 inches. When in the resting or open position, length c is about 1 inch and when in the closed or cutting position, length c is about 0.7 inches.

The apparatus 1 is comprised of a material that is suitably hard such that the cutting blades 8 do not become deformed by the formation of indentations, or have a piece break out of one or both jaws, upon cutting the metallic embolic coils. In addition, the cutting blades should be suitably sharp as to allow the user to easily and cleanly cut through metallic embolic coils. Suitable materials include titanium, stainless steel, or a steel alloy comprising iron with one or more additives such as carbon, manganese, nickel, phosphorus, sulfur, silicon, copper, lead, bismuth, aluminum, boron, tungsten, molybdenum, silver, vanadium or chromium, and the like. These elements and/or compounds thereof are used to improve strength and prevent corrosion.

The weight of the apparatus as disclosed herein should be such that the user need not strain and can easily maneuver the apparatus and cut the coil(s). The weight is dictated by the composition and size of the apparatus. It may be that the handle portion comprises a different metallic composition than the cutting portion, thus allowing various sizes to be constructed with the desired weight.

The apparatus disclosed herein is designed for cutting embolic coils. The diameter of embolic coil is based on the desired properties (e.g., size, strength) and/or the applications of the embolic coil. Typically, embolic coils have a diameter of from about 0.001 inches to about 0.005 inches, or alternatively, from about 0.0015 inches to about 0.005 inches, or from about 0.002 inches to about 0.003 inches, from about 0.00225 inches to about 0.003 inches. In certain embodiments, such as when the embolic coils are to be used for neurological applications, they may have a diameter of at most about 0.002 inches. Although it is contemplated that the apparatus disclosed herein can be used to cut any embolic coil, it is especially useful in cutting metallic embolic coils. Some embolic coils which can be cut using the apparatus disclosed herein include those comprised of one or more metals or metal alloys, such as platinum, a platinum alloy (e.g., a platinum-tungsten alloy), stainless steel, nitinol and combinations thereof. In some embodiments, the embolic coils are metallic embolics comprised of platinum, stainless steel, nitinol, or combinations thereof. Embolic coils can be formed from wires with a round cross-section or from wires with other cross sections (e.g., ribbon-shaped wires).

### 3. Methods of the Invention

Methods of the invention are directed to cutting an embolic coil during a microsurgical procedure in the brain or spine of a patient, wherein said method comprises cutting said coil with an apparatus capable of minimizing the possibility of damage to the brain or spine. To our knowledge, there is no surgical instrument capable of achieving this while limiting, or eliminating, the possibility for damage to the brain or spine.

The microsurgical procedure may include a surgical procedure on an aneurysm or vascular malformation which has been previously treated sub-optimally with metallic embolic coils, where the aneurysm or vascular malformation may need to be surgically clipped within the brain or spine of a patient. In such cases, surgical clipping of the aneurysm or vascular malformation may require a device capable of cutting the embolic coils prior to surgical clipping of the aneurysm or vascular malformation. In addition, the microsurgical procedure can comprise excision of an aneurismal sac. Ideally in these types of instances, the cutting would take place both quickly and cleanly in order to minimize the duration of the surgery as well as avoid any potentially dangerous debris from contaminating the patient and causing damage.

Methods of the invention are also directed to using the cutting apparatus described hereinabove to cut metallic embolic coils. One such embodiment comprises a method for cutting an embolic coil comprising: positioning an embolic coil between two cutting blades of an apparatus for cutting metallic embolic coils comprising first and second legs each having a cutting portion and a connecting portion wherein the legs are pivotally connected at a first pivot point between the cutting portion and the connecting portion; first and second handles which are pivotally connected at a second pivot point and each is pivotally connected to at least one leg in the connecting portion of the leg at a third and fourth pivot point and wherein all pivot points are substantially planar, wherein the cutting portion comprises cutting blades on the top and front of the cutting portion such that the cutting portions form a spoon element on the bottom, wherein the legs are adjustable by the handles between an open position and a closed position and further wherein, in said closed position, the cutting portions are capable of cutting embolic coils; and adjusting the handles such that the legs are in a closed position.

The methods disclosed herein can be performed either prior to or during a microsurgical procedure. In one embodiment, the apparatus disclosed herein is used for cutting metallic embolic coils during a microsurgical procedure. Examples of such microsurgical procedures include surgical clipping of an aneurysm or vascular malformation, the occlusion of a vessel, treatment of an aneurysm (e.g., an intracranial aneurysm), or treatment of a vascular malformation (i.e. arteriovenous malformation (AVM), fistula, etc.).

The methods disclosed herein are directed to cutting embolic coils. The diameter of embolic coil is based on the desired properties (e.g., size, strength) and/or the applications of the embolic coil. Typically, embolic coils have a diameter of from about 0.001 inches to about 0.005 inches, or alternatively, from about 0.0015 inches to about 0.005 inches, or from about 0.002 inches to about 0.003 inches, from about 0.00225 inches to about 0.003 inches. In certain embodiments, such as when the embolic coils are to be used for neurological applications, they may have a diameter of at most about 0.002 inches.

Although it is contemplated that the apparatus disclosed herein can be used to cut any embolic coil, it is especially useful in cutting metallic embolic coils. Some embolic coils which can be cut using the apparatus disclosed herein include those comprised of one or more metals or metal alloys, such as platinum, a platinum alloy (e.g., a platinum-tungsten alloy), stainless steel, nitinol and combinations thereof. In some embodiments, the embolic coils are metallic embolics comprised of platinum, stainless steel, nitinol, or combinations thereof. Embolic coils can be formed from wires with a round cross-section or from wires with other cross sections (e.g., ribbon-shaped wires). In some embodiments the coil is a platinum-containing embolic coil.

It is contemplated that force required to be applied to the apparatus in order to effectively cut a coil would be less than other available cutters due to the multiple pivot points.

It will be appreciated that those skilled in the art will be able to devise various arrangements which, although not explicitly described or shown herein, embody the principles of the invention and are included within its spirit and scope. Furthermore, all conditional language recited herein is principally intended to aid the reader in understanding the principles of the invention and the concepts contributed by the inventors to furthering the art, and are to be construed as being without limitation to such specifically recited conditions. Moreover, all statements herein reciting principles, aspects, and embodiments of the invention are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

We claim:

1. An apparatus for cutting embolic coils comprising
first and second legs each having a cutting portion and a connecting portion wherein the legs are pivotally connected at a first pivot point between the cutting portion and the connecting portion;
first and second handles which are pivotally connected at a second pivot point and each is pivotally connected to at least one leg in the connecting portion of the leg at a third and fourth pivot point and wherein all pivot points are substantially planar,
wherein the cutting portion comprises cutting blades on the top and front of the cutting portion such that the cutting portions form a spoon element on the bottom,
wherein the legs are adjustable by the handles between an open position and a closed position and further wherein, in said closed position, the cutting portions are capable of cutting embolic coils.

2. The apparatus of claim I, wherein upon cutting, the first and second pivot points remain substantially longitudinally planar and the third and fourth pair of pivots expand apart from each other and the longitudinal plane of the first and second pivot points.

3. The apparatus of claim 1, wherein the cutting portion of the first and second legs have a distal portion that is curved.

4. The apparatus of claim 1, wherein the first and second handles each contain a support arm located interiorly wherein each support arm is slidably connected.

5. The apparatus of claim 1, wherein the apparatus is comprised of stainless steel or titanium.

6. The apparatus of claim 1, wherein the embolic coils are metallic embodies comprised of platinum, stainless steel, nitinol, or combinations thereof.

7. The apparatus of claim 1, wherein the apparatus is a microsurgical device.

8. A method for cutting an embolic coil comprising positioning an embolic coil between two cutting blades of the apparatus of claim 1 and adjusting the handles such that the legs are in a closed position.

9. The method of claim 8, wherein the cutting is performed during a microsurgical procedure in the brain or spine of a patient.

10. The method of claim 9, wherein the cutting is performed while minimizing the possibility of damage to the brain or spine during a microsurgical procedure in a patient.

11. The method of claim 10, wherein the microsurgical procedure comprises treating an aneurysm.

12. The method of claim 10, wherein the microsurgical procedure comprises treating a vascular malformation.

13. A method for cutting an embolic coil during an microsurgical procedure in the brain or spine of a patient, wherein said method comprises cutting said coil with an apparatus capable of minimizing the possibility of damage to the brain or spine.

14. The method of claim 13, wherein the microsurgical procedure is in the brain of the patient.

15. The method of claim 13, wherein the microsurgical procedure is in the spine of the patient.

16. The method of claim 13, wherein the microsurgical procedure comprises treating an aneurysm.

17. The method of claim 13, wherein the microsurgical procedure comprises treating a vascular malformation.

18. The method of claim 13, wherein the apparatus is the apparatus of any one of claims 1 to 7.
